(19) United States
(12) Patent Application Publication
(10) Pub. No.: US 2025/0134967 A1
(43) Pub. Date: May 1, 2025

(54) COMPOSITIONS COMPRISING ACID ALPHA GLUCOSIDASE AND METHODS OF USE THEREOF

(71) Applicant: M6P THERAPEUTICS, INC., St. Louis, MO (US)

(72) Inventors: Robert Russell GOTSCHALL, Doylestown, PA (US); Lin LIU, Ballwin, MO (US)

(21) Appl. No.: 18/835,767

(22) PCT Filed: Feb. 7, 2023
(86) PCT No.: PCT/US2023/012520

§ 371 Date: Aug. 5, 2024

(51) Int. Cl. A61K 38/47; A61P 3/00; C12N 9/24

(52) U.S. Cl. CPC A61K 38/47; A61P 3/00; C12N 9/2402; C12Y 302/0102

(57) ABSTRACT

Provided are compositions comprising acid alpha glucosidase (GAA) having increased phosphorylation of High Mannose N-linked Oligosaccharides. Also provided herein are methods of treating Pompe disease comprising administering to a subject the compositions of the disclosure.

### CROSS REFERENCE TO RELATED APPLICATIONS

This application is a 371 of International Application No. PCT/US2023/12520, filed Feb. 7, 2023, which claims the benefit of priority U.S. Patent Application Ser. No. 63/307,456, filed Feb. 7, 2022, the entire contents of which are incorporated herein by reference.

### BACKGROUND

Lysosomes are cellular organelles with an acidic interior containing a wide variety of hydrolytic enzymes that break down macromolecules such as nucleic acids, proteins, and polysaccharides. Lysosomal storage disorders (LSDs) relate to inherited metabolic disorders that result from defects in lysosomal function. Currently, about 50 distinct LSDs have been identified but a small number of these (fewer than 10) are reported to have treatments.

Pompe disease is a rare autosomal recessive disorder in which toxic levels of the complex sugar, glycogen, accumulates in cells. See, e.g., Fukuda T et al., Acid alpha-glucosidase deficiency (Pompe disease). Curr Neurol Neurosci Rep. 2007 January; 7(1):71-7. doi: 10.1007/s11910-007-0024-4. PMID: 17217857. Pompe disease is caused by low levels or absence of acid alpha glucosidase (GAA) activity, an enzyme that normally breaks down glycogen in the lysosome. GAA deficiency results in glycogen accumulating in lysosomes in multiple tissues, particularly cardiac and skeletal muscle.

Currently there are no curative therapies for Pompe disease. Instead, enzyme replacement therapy (ERT) is used to replace the deficient/absent GAA with a substitute (e.g., alglucosidase alfa), which allows the body to break down the glycogen that otherwise builds up. Current approaches to ERT, however, have drawbacks including, for example, poor cellular uptake (particularly in cardiac and skeletal muscles) and tissue penetration (requiring higher dosing), long and frequent infusions, immunogenicity, and the like. Therefore, there is a need for safe and effective treatments for Pompe disease.

### SUMMARY

The present disclosure is directed to a composition comprising acid alpha glucosidase, wherein at least 50% of the acid alpha glucosidase comprises at least one phosphorylated mannose group.

In some embodiments, at least 25% of the acid alpha glucosidase comprises bis-phosphorylated mannose groups. In some embodiments, at least 50% of the acid alpha glucosidase comprises bis-phosphorylated mannose groups. In some embodiments on average each acid alpha glucosidase molecule comprises at least 2 bis-phosphorylated N-linked glycan.

In any of the foregoing embodiments, less than 8% of the acid alpha glucosidase glycans comprises non-phosphorylated N-linked glycans.

In any of the foregoing embodiments, less than 40% of the glycans found on acid alpha glucosidase comprises complex type N-glycans.

In any of the foregoing embodiments, less than 3% of the acid alpha glucosidase oligosaccharides comprises hybrid type N-glycans.

In any of the foregoing embodiments, the composition comprises less than 5 nanomolar affinity for CIMPR.

In any of the foregoing embodiments, composition comprises from about 0.1 to 5-fold greater ability to reduce glycogen compared to alglucosidase alfa.

In any of the foregoing embodiments, the composition comprises from about 0.1 to 5-fold greater ability to reduce glycogen compared to alglucosidase alfa in at least one tissue selected from the group consisting of heart, quad, triceps, diaphragm, or psoas.

In any of the foregoing embodiments, acid alpha glucosidase is capable of being endogenously hydrolyzed.

The present disclosure is also directed to a method of treating Pompe disease, the method comprising administering to a subject an effective amount of the composition of any of the foregoing embodiments. In some embodiments, administering comprises enzyme replacement therapy.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIG. 1A depicts a CIMPR affinity chromatography profile of M021 produced in HEK293 cells.

FIG. 1B depicts a CIMPR affinity chromatography profile of M021 produced in CHO cells.

FIG. 1C depicts a CIMPR affinity chromatography profile of alglucosidase alfa.

FIG. 1D depicts a CIMPR affinity chromatography profile of ATB200.

FIG. 1E depicts CIMPR binding affinity determination of M021 expressed in CHO cells compared to alglucosidase alfa.

FIG. 2 depicts half-life of M021 and alglucosidase alfa.

FIG. 3 depicts GAA activity of M021 and alglucosidase alfa in heart, quad, and triceps following enzyme replacement therapy.

FIG. 4 depicts percent N-glycan types in preparations of M021, ATB200, and alglucosidase alfa and 2AB glycan profiling of M021 produced in either HEK293 or CHO cells.

FIG. 5 depicts the average number of bis-phosphorylated mannose groups per GAA molecule in preparations of M021, ATB200, and alglucosidase alfa.

FIG. 6 depicts tissue GAA activity and glycogen levels of M021 and alglucosidase alfa treated Gaa KO mice.

FIG. 7 depicts Periodic-Acid Schiff (PAS) stains of quadriceps tissue in Gaa KO mice treated with M021 or alglucosidase alfa.

FIG. 8 depicts glycogen levels in heart tissue in mice treated with M021 or alglucosidase alfa.

FIG. 9 depicts PAS stains of heart tissue in mice treated with M021 or alglucosidase alfa.

FIG. 10 depicts glycogen levels in quadriceps tissue in mice treated with M021 or alglucosidase alfa.

FIG. 11 depicts PAS stains of quadriceps tissue in mice treated with M021 or alglucosidase alfa.

FIG. 12 depicts glycogen levels in triceps tissue in mice treated with M021 or alglucosidase alfa.

FIG. 13 depicts PAS stains of triceps tissue in mice treated with M021 or alglucosidase alfa.

FIG. 14 depicts glycogen levels in diaphragm tissue in mice treated M021 or alglucosidase alfa.

FIG. 15 depicts glycogen levels in psoas tissue in mice treated M021 or alglucosidase alfa.

FIG. 16 depicts glycogen levels in brain tissue in mice treated with M021 or alglucosidase alfa.

FIG. 17 depicts glycogen levels in heart, quadriceps, triceps, and diaphragm tissue in mice treated with M021 or alglucosidase alfa.

FIG. 18 depicts grip strength of mice treated with M021 or alglucosidase alfa in a five-month ERT study.

FIG. 19 depicts grip strength of mice treated with M021 or alglucosidase alfa in a six-month ERT study.

FIG. 20A depicts reduced glycogen in the heart of M021-treated mice.

FIG. 20B depicts reduced glycogen in the quad of M021-treated mice.

FIG. 20C depicts reduced glycogen in the diaphragm of M021-treated mice.

FIG. 20D depicts reduced glycogen in the triceps of M021-treated mice.

FIG. 20E depicts reduced glycogen in the psoas of M021-treated mice.

### DETAILED DESCRIPTION

The current invention is directed to novel compositions comprising GAA having increased phosphorylation of High Mannose N-linked Oligosaccharides and methods of use thereof.

### Definitions

Unless defined otherwise, all technical and scientific terms used herein have the same meaning as commonly understood by one of ordinary skill in the art to which the invention pertains. In describing and claiming the present invention, the following terminology will be used.

As used herein, the terms “N-linked glycan,” “mannose group,” “N-linked oligosaccharide,” and the like are interchangeable.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, com oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the vims particle or infectious units generated from vector of the invention which is required to prevent the particular disease condition, or which reduces the severity of and/or ameliorates the disease condition or at least one symptom thereof or condition associated therewith.

### Compositions of the Disclosure

The disclosure provides a composition comprising GAA having increased phosphorylation of High Mannose N-linked Oligosaccharides.

Lysosomal proteins, like GAA, are modified by N-glycosylation, which refers to transfer of a GlcNAc2-Man9-Glc3 structure to the Asn-X-Ser/Thr (x any amino acid except His or Pro) motif. These glycoconjugates contain a GlcNAc2 mannose (Man) 3 core. From there, the glycoconjugates are processed. Processing can include, for example, phosphorylation, trimming and rebuilding. The structure may be classed as a high-mannose N-glycan, a hybrid N-glycan or a complex N-glycan. Over 130 different N-linked glycan structures are possible.

Additionally, the N-glycan may be phosphorylated to contain one or more mannose 6-phosphate (M6P). For example, a N-glycan on a lysosomal protein molecule may bear one M6P group (mono-phosphorylated) or two M6P groups (bis-phosphorylated) on a single glycan structure. The M6P serves as a cellular signal, directing lysosomal proteins to lysosomes—their site of activity—through membrane bound receptors (e.g., cation-independent mannose 6-phosphate receptor or CIMPR). The number of M6P groups affects the binding affinity to CIMPR—bis-phosphate has a higher biding affinity than mono-phosphate forms.

### M6P Content

The GAA according to the present disclosure comprises a high percentage of bis-phosphorylated M6P. In some embodiments, at least 25% of the N-linked oligosaccharides on the GAA are bis-phosphorylated. For example, at least 25%, 30%, 35%, 40%, 45%, 50%, 55%, 60%, or 65% of the N-linked glycans on the GAA are bis-phosphorylated.

The GAA according to the present disclosure comprises a higher percentage of phosphorylated N-linked oligosaccharides a corresponding lower percentage of non-phosphorylation phosphorylation-linked oligosaccharides. In some embodiments, less than 10% of the N-linked glycans on the GAA are not phosphorylated. For example, less than 10%, 9%, 8%, 7%, 6%, 5%, 4%, 3%, 2%, or 1% of the N-linked glycans on the GAA are non-phosphorylated. In some embodiments, at least 40% of the N-linked glycans on the GAA are phosphorylated. For example, at least 40%, 45%, 50%, 55%, 60%, 65%, 70%, 75%, 80%, 85%, 90%, 95%, 96%, 97%, 98%, or 99% of the N-linked glycans on the GAA are phosphorylated. In some embodiments, at least 15% of the N-linked glycans on the GAA are mono-phosphorylated. For example, at least 15%, 20%, 25%, or 30% of the N-linked on the GAA are mono-phosphorylated.

In a preferred embodiment, less than 5% of the N-linked glycans on the GAA are non-phosphorylated and at least 40% of the phosphorylated N-linked glycans on the GAA are bis-phosphorylated.

In some embodiments, on average the N-glycans contain greater than 0.25 mol/mol of bis-M6P. For example, on average the N-glycans contain greater than 0.25 mol/mol, 0.5 mol/mol, 0.75 mol/mol, 1 mol/mol, 1.25 mol/mol, 1.5 mol/mol, 1.75 mol/mol, 2 mol/mol, 2.25 mol/mol, 2.5 mol/mol, 2.75 mol/mol, 3 mol/mol, 3.25 mol/mol, 3.5 mol/mol, 3.75 mol/mol, 4 mol/mol, 4.25 mol/mol, 4.5 mol/mol, 4.75 mol/mol, 5 mol/mol, 5.5 mol/mol, or 6 mol/mol of bis-M6P. In a preferred embodiment, the GAA according to the present disclosure comprises on average at least 3 moles of bis-M6P per mole of GAA.

### Glycan Type

The GAA according to the present disclosure comprises a low level of neutral N-linked glycans. In some embodiments, the GAA comprises less than 5% neutral N-linked glycans, which provides reduced rates of non-productive clearance via mannose receptors and asialoglycoprotein receptors and improved ability to target cells.

The GAA according to the present disclosure comprises a reduced percentage of complex and hybrid-type N-glycans. In some embodiments, less than 50% of the total N-glycans on the GAA are complex type N-glycans. For example, less than 50%, 45%, 40%, 35%, 30%, 25%, 20%, 15%, 10%, or 5% of the total N-glycans on the GAA are complex type N-glycans. In some embodiments, less than 5% of the total N-glycans on the GAA are hybrid type N-glycans. For example, less than 5%, 4%, 3%, 2%, or 1% of the total N-glycans on the GAA are hybrid type N-glycans. In a preferred embodiment, less than 20% of the total N-glycans on the GAA are complex type N-glycans and less than 1% of the total N-glycans on the GAA are hybrid type N-glycans.

### Glycosylation Profile

GAA has seven N-glycosylation sites. The GAA according to the present disclosure comprises an improved glycosylation profile.

In some embodiments, at least 10% of GAA is phosphorylated at the first N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the first N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the first N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the first N-glycosylation site.

In some embodiments, at least 10% of GAA is phosphorylated at the second N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the second N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the second N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the second N-glycosylation site.

In some embodiments, at least 10% of GAA is phosphorylated at the third N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the third N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the third N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the third N-glycosylation site.

In some embodiments, at least 10% of GAA is phosphorylated at the fourth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the fourth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the fourth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the fourth N-glycosylation site.

In some embodiments, at least 10% of GAA is phosphorylated at the fifth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the fifth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the fifth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the fifth N-glycosylation site.

In some embodiments, at least 10% of GAA is phosphorylated at the sixth N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the sixth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the sixth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the sixth N-glycosylation site.

In some embodiments, at least 10% of GAA is phosphorylated at the seventh N-glycosylation site. For example, at least 10%, 15%, 20%, 25%, 30%, 35%, 40%, 45%, 50%, 55%, 60%, 65%, 70%, 75%, 80%, 85%, 90%, or 95% of the GAA can be phosphorylated at the seventh N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the GAA bears a mono-M6P unit at the seventh N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the GAA bears a bis-M6P unit at the seventh N-glycosylation site.

### CIMPR Binding and Cell Uptake

The GAA according to the present disclosure comprises increased cation independent mannose 6-phosphate receptor (CIMPR) binding and cellular uptake due to increased phosphorylation. In some embodiments, the GAA has less than 5 nanomolar affinity for the CIMPR. For example, the GAA has less than 5 nanomolar, 4 nanomolar, 3 nanomolar, 2 nanomolar, or 1 nanomolar for the CIMPR.

In some embodiments, at least 30% of the GAA can interact with the CIMPR. For example, at least 30%, 35%, 40%, 45%, 50%, 55%, 60%, 65%, 70%, 75%, 80%, 85%, 90%, or 95% of the GAA can interact with the CIMPR.

In a preferred embodiment, the GAA has less than 4 nanomolar affinity for the CIMPR and at least 75% of the GAA can interact with CIMPR.

### Ability to Reduce Glycogen

In some embodiments, the GAA of the present invention is more effective at reducing glycogen content in cells. For example, the GAA of the present invention may be about 0.1 to 5-fold greater reduction of substrate compared to alglucosidase alfa.

In some embodiments, the GAA of the present invention is more effective at reducing substrate compared to alglucosidase alfa in at least one of heart, quad, triceps, diaphragm, and psoas.

Enzymatic Phosphorylation. Phosphorylation of the GAA of the present disclosure is achieved enzymatically, not chemically, so the GAA is capable of being degraded endogenously in the lysosome. A non-limiting example of compounds and compositions that can be endogenously hydrolyzed include compounds and compositions that lack a hydrazone bond. Unlike unnatural chemical bonds like hydrazone bonds that are typically used for chemical conjugation of synthetic glycans, the GAA of the present disclosure comprises natural glycosidic bonds that can be properly processed in lysosomes after cellular uptake in target cells.

### Pharmaceutical Compositions and Formulations

Also provided herein is a pharmaceutical composition comprising GAA of the disclosure.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the presently disclosed compositions can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In some embodiments, the disclosure provides a pharmaceutical composition for treating a subject suffering from Pompe disease.

Pharmaceutically acceptable carriers, which are useful, include, but are not limited to, glycerol, water, saline, ethanol and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The disclosed composition may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. In some embodiments, the preservative is a combination of about 0.5% to 2.0% benzyl alcohol and 0.05% to 0.5% sorbic acid.

The composition may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. In some embodiments, BHT and disodium edetate are the antioxidant and the chelating agent respectively for some compounds, however, other suitable and equivalent antioxidants and chelating agents may be substituted therefore as would be known to those skilled in the art.

The disclosure provides a pharmaceutical composition comprising GAA of the disclosure and a pharmaceutically acceptable carrier.

The compositions of the disclosure may be used for enzyme replacement therapy (ERT). Alternatively or in addition, the compositions of the disclosure may be used for gene therapy.

### Methods of the Disclosure

The disclosure provides method to treat a subject deficient for GAA activity with Pompe disease or in a subject at risk for developing Pompe disease. The method introduces phosphorylated GAA thereby treating the subject or preventing the occurrence of Pompe disease in the subject. Further, the method improves quality of life in a patient.

The disclosure provides a method of treating Pompe disease, the method comprising administering to a subject a composition of the disclosure, thereby treating Pompe disease.

The disclosure provides a method of treating Pompe disease, the method comprising administering to a subject a therapeutically effective amount of a composition of the disclosure, thereby treating Pompe disease.

The disclosure provides a method of treating a subject suffering from Pompe disease, the method comprising administering to the subject a pharmaceutical composition of the disclosure, treating the subject.

The disclosure provides a method of preventing the occurrence of Pompe disease in a subject in need thereof, the method comprising administering to the subject a pharmaceutical composition of the disclosure, thereby preventing the occurrence of Pompe disease in the subject.

### Administration/Dosing

The regimen of administration may affect what constitutes an effective amount. For example, the therapeutic formulations may be administered to the patient subject either prior to or after a surgical intervention related to Pompe disease, or shortly after the patient was diagnosed with Pompe disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient subject, preferably a mammal, more preferably a human, may be carried out using known procedures, at dosages and for periods of time effective to treat Pompe disease in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non limiting example of an effective dose range for a therapeutic compound of the disclosure is from about 0.01 and 50 mg/kg of body weight/per day.

The composition can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of Pompe disease.

### Routes of Administration

One skilled in the art will recognize that although more than one route can

be used for administration, a particular route can provide a more immediate and more effective reaction than another route.

Routes of administration of the disclosed compositions include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, intra-cisterna magna (ICM), intraspinal, intraventricular, intracerebroventricular, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein. In one embodiment, the treatment of Pompe disease comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal and any combination thereof.

Furthermore, the actual dose and schedule can vary depending on whether the compositions are administered in combination with other pharmaceutical compositions, or depending on interindividual differences in pharmacokinetics, drug disposition, and metabolism. One skilled in the art can easily make any necessary adjustments in accordance with the exigencies of the particular situation.

### EXAMPLES

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1: Transient Expression of a Bicistronic hGAA+S1S3 PTase in Expi293 and CHO Cell Lines

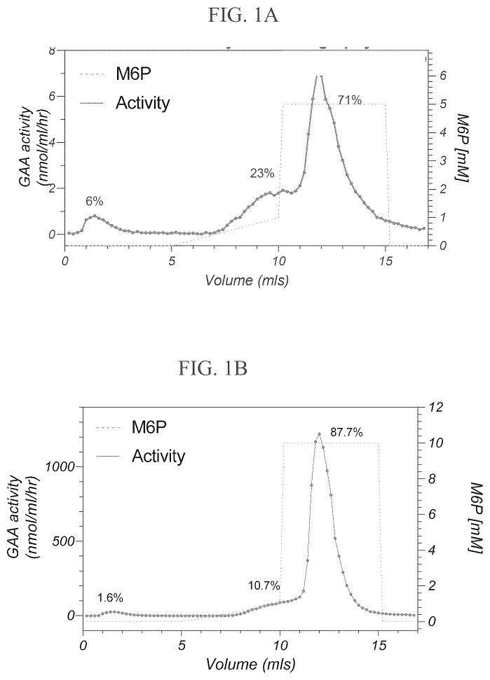

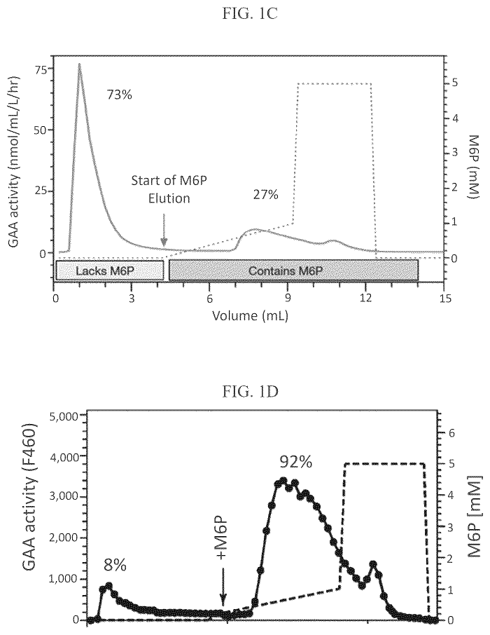

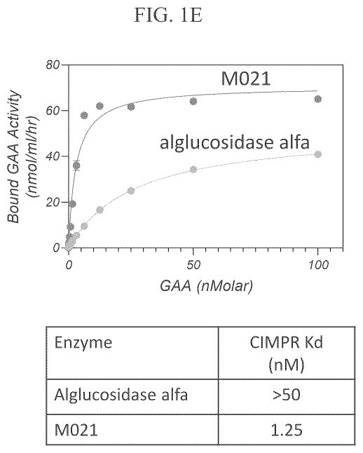

GAA was transiently co-expressed in Expi293 or CHO cells utilizing a bicistronic vector encoding the S1S3 variant of N-acetylglucosamine-1-phosphate transferase (PTase) (see Lin Liu et al. Engineering of GlcNAc-1-Phosphotransferase for Production of Highly Phosphorylated Lysosomal Enzymes for Enzyme Replacement Therapy, Mol Ther Methods Clin Dev. 2017 Jun. 16; 5:59-65) and GAA (M021), followed by purification of the M021. As demonstrated in FIGS. 1A-1E, compared to alglucosidase alfa and ATB200, M021 has greater amounts of enzyme that can bind the CIMPR as measured by CIMPR affinity chromatography. In Expi293 and CHO cells expressing the bicistronic vector and GAA, phosphorylated M6P comprised at least 90% (FIG. 1A, Expi293) (FIG. 1B, CHO), compared to around 27% of alglucosidase alfa comprising phosphorylated M6P (FIG. 1C). FIG. 1D shows that while the same amount of ATB200 is able to bind the affinity column, the amount of M6P that is required to compete it off is much lower. The majority of ATB200 is mainly eluted with up to 1 mM M6P while the majority of M021 requires at least 5 mM M6P to elute, indicating greater phosphorylation. Moreover, M021 expressed in CHO cells comprises a higher CI-MPR binding affinity of (Kd=1.25 nM) compared to a CI-MPR binding affinity (Kd=>50 mM) for alglucosidase alfa (see FIG. 1E).

### Example 2: Single Dose PK/PD Study in GAAKO Pompe Mouse Model

A PK/PD study in the GAAKO Pompe mouse model was performed using the Pompe disease standard of care, alglucosidase alfa, and purified M021 (as described in Example 1). Briefly, 12-14 week old mice (n=5 per group) received a single dose of alglucosidase alfa or M021 at 0.2, 2, or 20 mg/kg dosage. At 2, 30, 60 and 120 mins, serum samples were collected to measure GAA activity and 24 hours later tissues were collected and activity of GAA was assessed and IHC performed (data not shown).

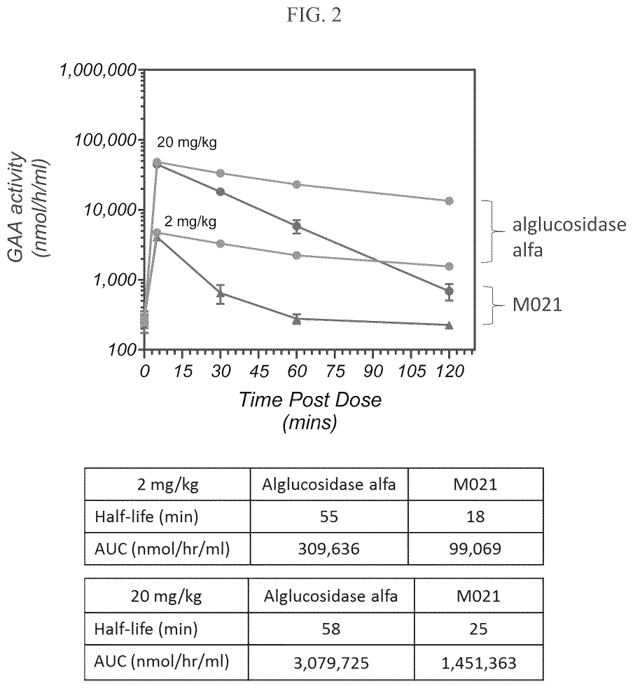

As can be seen in FIG. 2, M021 at both 2 mg/kg and 20 mg/kg dosages has a shorter half-life compared to alglucosidase alfa.

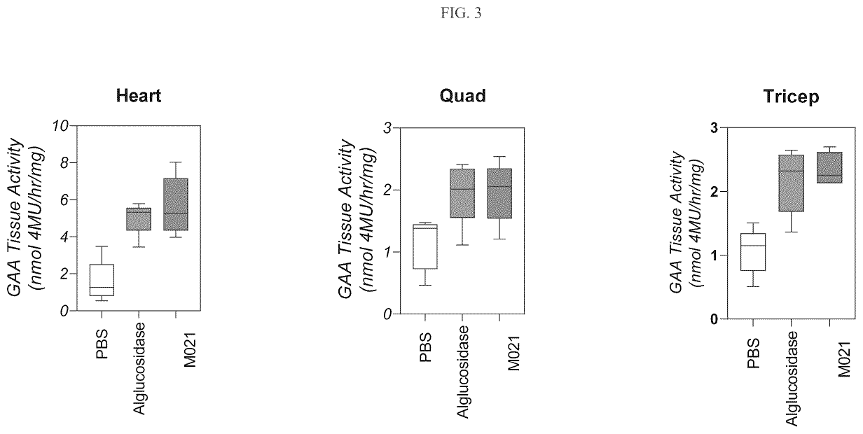

As can be seen in FIG. 3, despite having a shorter half-life and lower AUC, the same amount of GAA is found in heart, quad, and triceps tissue.

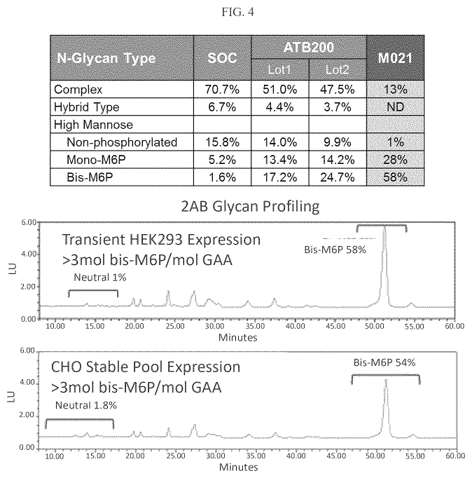

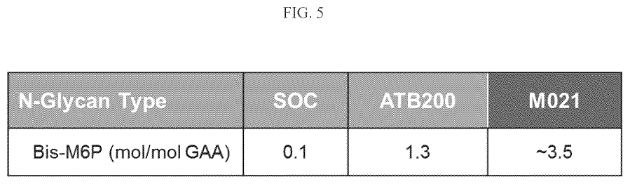

To assess glycan characteristics, GAA was reduced and alkylated, treated with PNGaseF to release the glycan, labeled with 2AB fluorescent probe and separated by HPLC. Glycan peaks are proportional to one another since a single probe is added to each glycan. Glycan peaks were identified by MS/MS. Compared to standard of care Lumizyme/alglucosidase alfa and two lots of ATB200 (CA3010205A1-Augmented acid alpha-glucosidase for the treatment of Pompe disease), M021 contains fewer complex and hybrid-type N-glycans, fewer non-phosphorylated mannose groups, more mono-phosphorylated mannose groups, and more bis-phosphorylated mannose groups (see FIG. 4). M021 has on average greater than 3 bis-phosphorylated mannose groups per GAA molecule, more than both the standard of care, alglucosidase alfa, which has less than 1 bis-phosphorylated mannose group per GAA molecule, and ATB200, which has around 1 bis-phosphorylated mannose group per GAA molecule (see FIG. 5).

### Example 3: Two Dose ERT Study in GaaKO Pompe Mouse Model

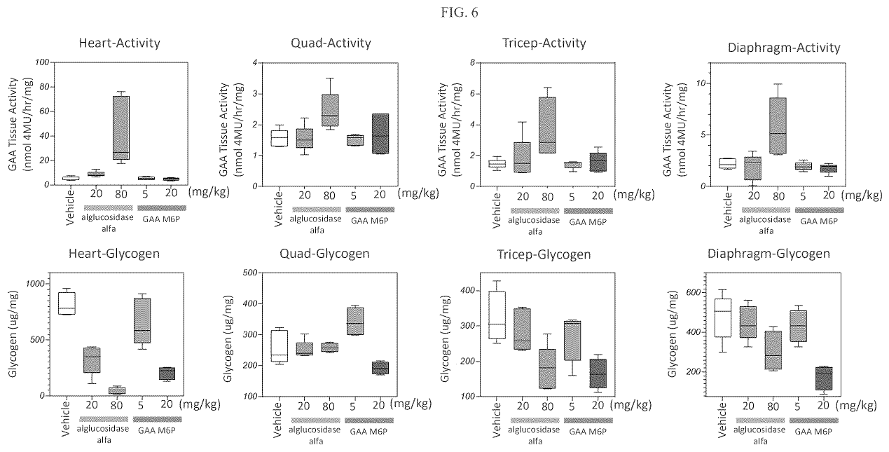

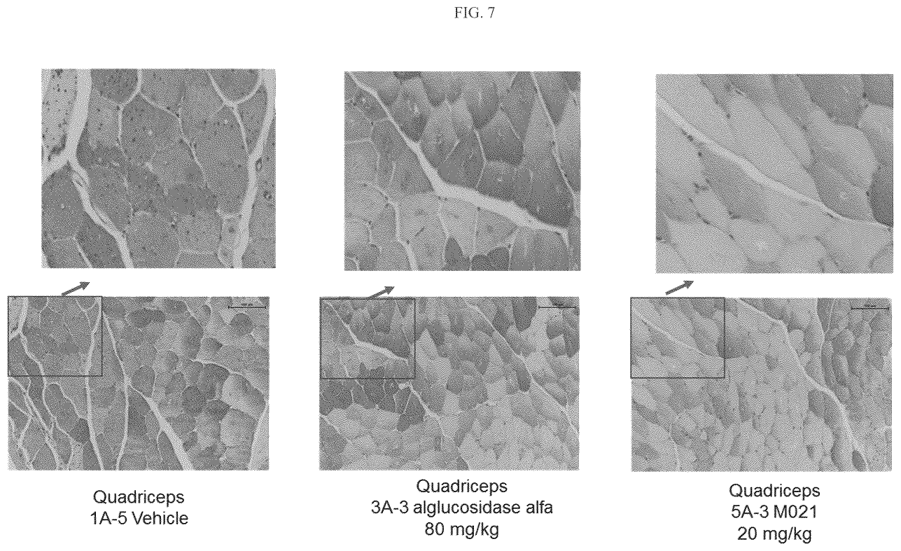

A two-dose efficacy study in the GaaKO Pompe mouse model was performed using the Pompe disease standard of care, alglucosidase alfa, and purified M021 (as described in Example 1). Briefly, 12-14 week old mice (n=5 per group) received two doses of alglucosidase alfa (20 or 80 mg/kg dosages) or M021 (5 or 20 mg/kg dosages) on day 1 and day 14, with evaluation of GAA activity, presence of substrate glycogen, PAS staining, and IHC performed at day 23. As shown in FIG. 6, M021 activity is not significantly greater than alglucosidase alfa in any of the tissues assessed, however, M021 at a dosage of 20 mg/kg is better at reducing cellular glycogen compared to alglucosidase alfa at 80 mg/kg (the standard of care dosage). As seen in FIG. 7, PAS stains of quadriceps tissue in mice treated with 20 mg/kg M021 show more even reduction of glycogen across all cells compared to mice treated with 80 mg/kg alglucosidase alfa.

### Example 4: Two Dose Efficacy Study With M021

GAA was transiently co-expressed in CHO cells utilizing a bicistronic vector encoding the S1S3 variant of N-acetylglucosamine-1-phosphate transferase (PTase), followed by purification of the M021 with an affinity tag followed by anion exchange chromatography to eliminate neutral glycosylated GAA and enrich for phosphorylated and well sialylated glycans to generate M021.

A two-dose efficacy study with optimized oligosaccharides in the GaaKO Pompe mouse model was performed using the Pompe disease standard of care, alglucosidase alfa, and purified M021 as described above. Briefly, 12-14 week old mice (n=6 per group) received two doses of alglucosidase alfa (20 mg/kg dosage) or M021 (5, 10, or 20 mg/kg dosages) on day 1 and about day 14, with evaluation of GAA activity, presence of substrate glycogen, PAS staining, and IHC performed 14 days following the second dose.

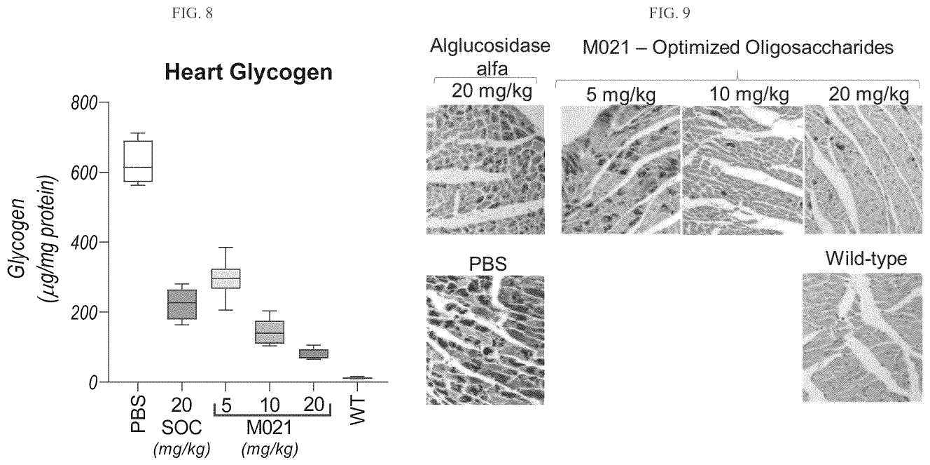

Heart. As shown in FIGS. 8, M021 at 10 and 20 mg/kg dosages is significantly better at clearing glycogen in the heart compared to alglucosidase alfa at 20 mg/kg. As shown in FIG. 9, PAS stains of heart tissue show that M021 is better at clearing glycogen and resolving cell pathology.

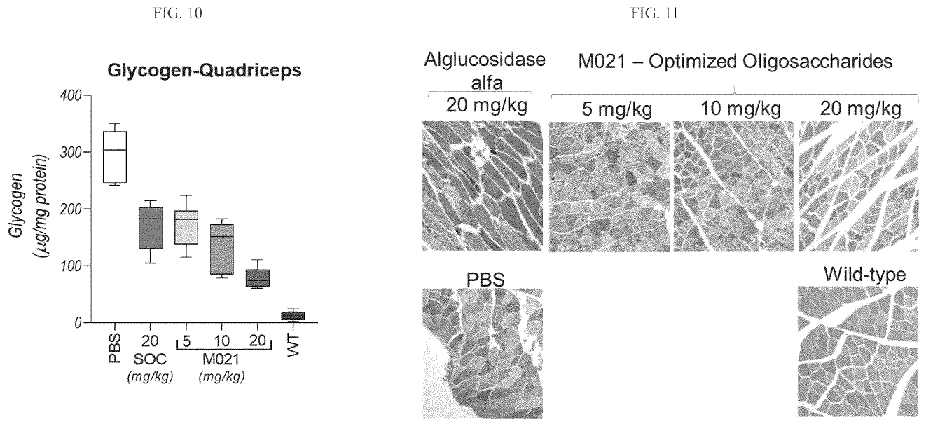

Quadricep. As shown in FIGS. 10, M021 at 10 and 20 mg/kg dosages is significantly better at clearing glycogen in the quadricep compared to alglucosidase alfa at 20 mg/kg. As shown in FIG. 11, PAS stains of quadricep tissue show that M021 is better at clearing glycogen and resolving cell pathology.

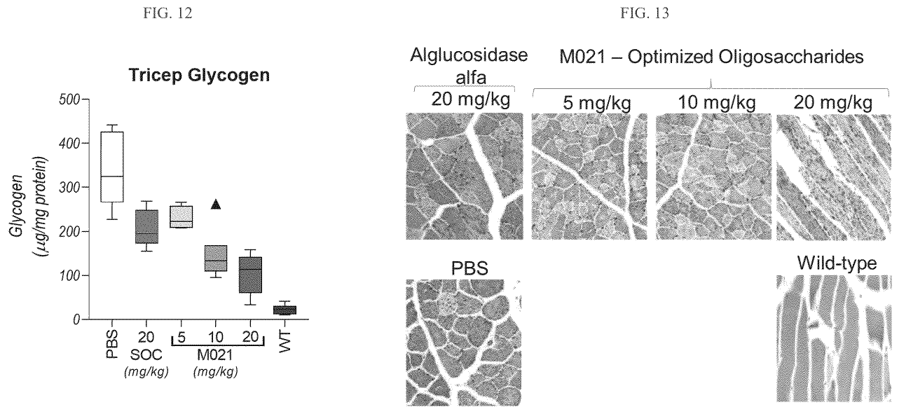

Tricep. As shown in FIGS. 12, M021 at 10 and 20 mg/kg dosages is significantly better at clearing glycogen in the triceps compared to alglucosidase alfa at 20 mg/kg. As shown in FIG. 13, PAS stains of triceps tissue show that M021 is better at clearing glycogen and resolving cell pathology.

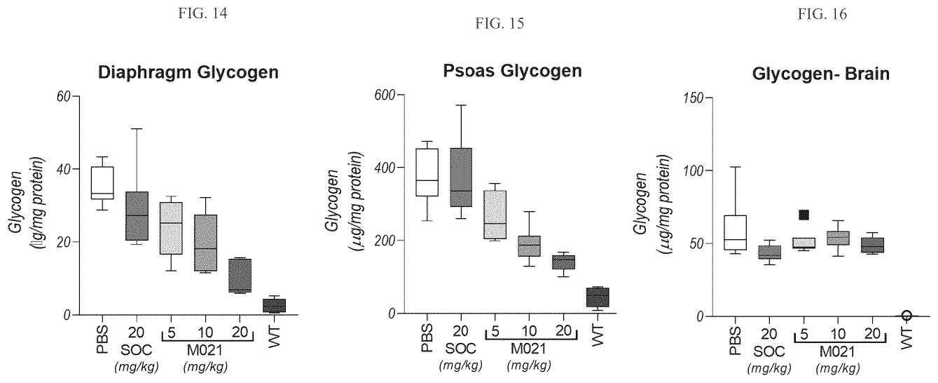

Diaphragm. As shown in FIG. 14, M021 at 20 mg/kg dosages is significantly better at clearing glycogen in the diaphragm compared to alglucosidase alfa at 20 mg/kg.

Psoas. As shown in FIGS. 15, M021 at 10 and 20 mg/kg dosages is significantly better at clearing glycogen in the psoas compared to alglucosidase alfa at 20 mg/kg.

Brain. As shown in FIG. 16, neither alglucosidase alfa nor M021 are able to significantly reduce glycogen in the brain.

### Example 5: Six-Month ERT Study in GaaKO Pompe Mouse Model

A long-term efficacy study in the GaaKO Pompe mouse model was performed using the Pompe disease standard of care, alglucosidase alfa, and purified M021 as described in Example 4. Briefly, 12-14 week old mice (n=6 per group) received eight doses of alglucosidase alfa (20 mg/kg dosage) or M021 (5, 10, or 20 mg/kg dosages) over the course of six months, with evaluation of GAA activity, presence of substrate glycogen, PAS staining (data not shown), and IHC (data not shown) performed at month seven and in-life tests of grip strength performed during the course of treatment. For the grip-strength test, an axial force transducer grip meter designed for mice was used. Each mouse was held by the base of the tail and lowered toward a trapeze (triangle bar) mounted to the grip meter. Mice were allowed to grasp the bar with both forepaws and were then pulled by the tail away from the rod in one fluid motion. The maximal lateral force exerted on the gauge was recorded. Each mouse was assessed with 5consecutive trials in 1 day and the average value is reported.

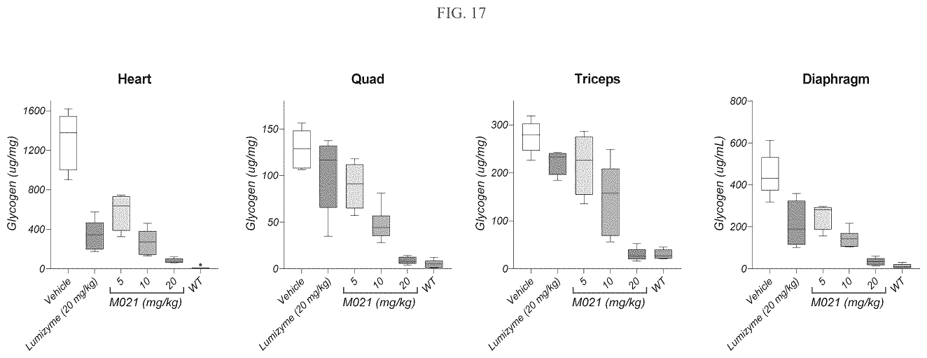

As shown in FIG. 17, M021 is better at clearing glycogen in the heart, quad, triceps, and diaphragm compared to alglucosidase alfa/Lumizyme.

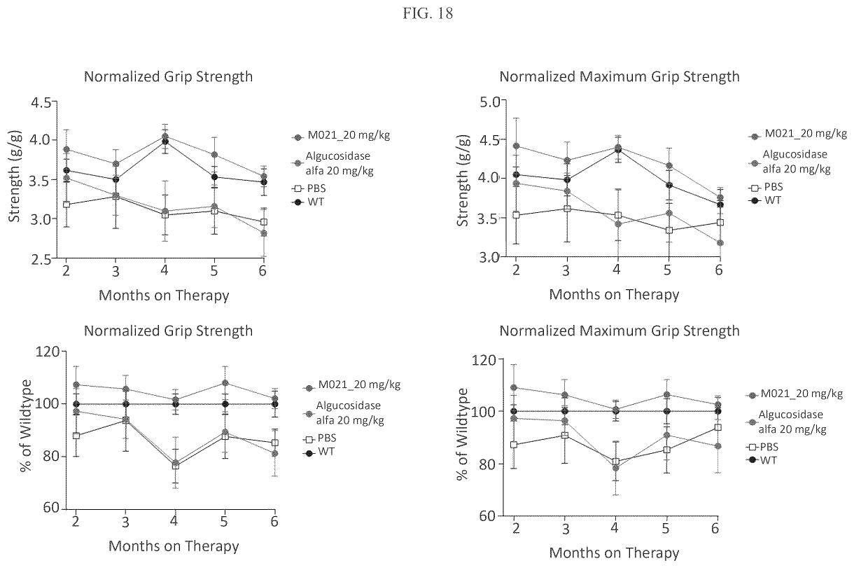

Data presented in FIG. 18 shows mice treated with M021 exhibit improved grip strength compared to mice treated with alglucosidase alfa.

### Example 6: Six Month ERT Study in GaaKO Pompe Mouse Model

A long-term efficacy study in the GaaKO Pompe mouse model was performed using the Pompe disease standard of care, alglucosidase alfa, and purified M021 as described in Example 4. Briefly, 10-12 week old mice (n=5-11 per group, 8 groups, 64 mice total) received bi-weekly doses for 8 doses (4 months total) or 13 doses (6.5 months total) of alglucosidase alfa (20 mg/kg dosage) or M021 (20 mg/kg dosage), with evaluation of GAA activity and presence of substrate glycogen performed at 14 days post last dose. In-life tests of grip strength was performed during the course of treatment. For the grip-strength test, a dual sensor grip strength meter designed for mice was used. Each mouse was held by the base of the tail and allowed to grasp the pull bar assembly mounted to the meter with the forelimbs. The mouse was then pulled back in the horizontal plane until the mouse released the grip. The maximal peak force applied to the bar was displayed and recorded. Each mouse was assessed with 5 consecutive trials in 1 day and the average value is reported.

Data presented in FIG. 19 shows mice treated with M021 exhibit improved grip strength compared to mice treated with alglucosidase alfa. Specifically, mice treated with M021 show the same forelimb grip strength as wild type mice starting 2 months after treatment began whereas mice treated with alglucosidase alfa showed no significant improvement over mice treated with vehicle (FIG. 19).

### Example 7: Reduction of Glycogen in Aged GaaKO Pompe Mouse Model

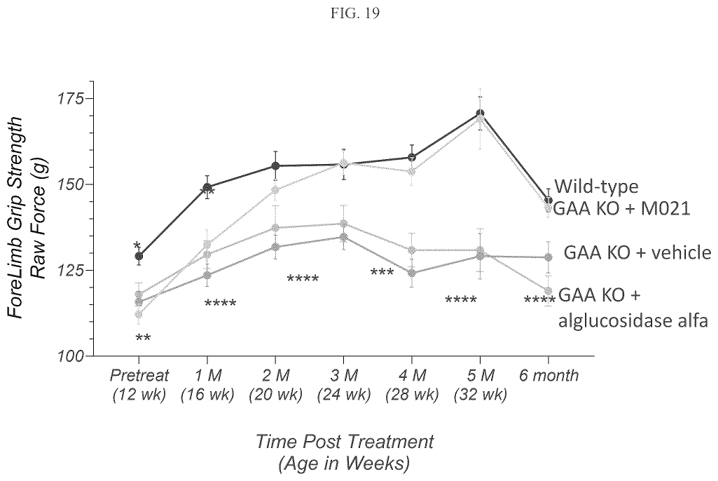

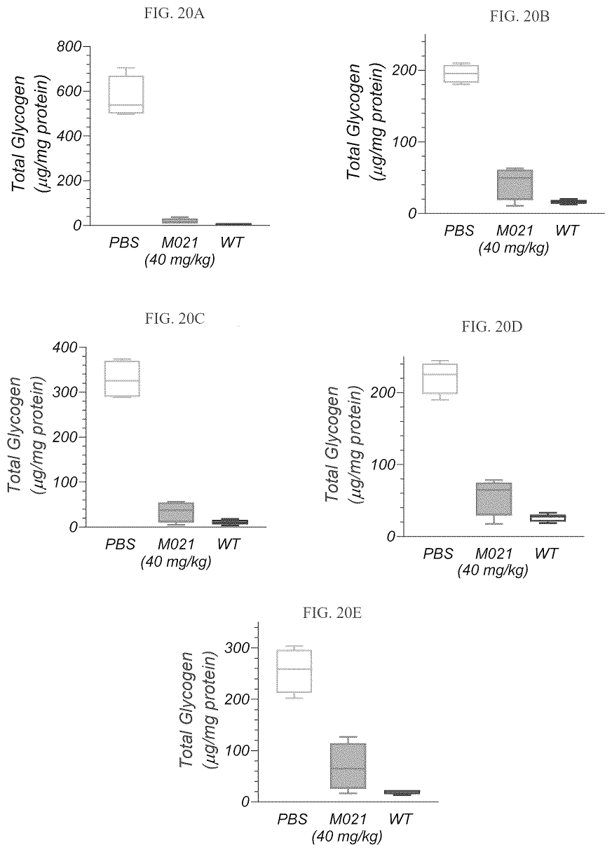

12-month female GaaKO mice (n=8) received weekly M021 (40 mg/kg dosage, n=4) or no treatment (n=4), and 5 approximately age matched wild type mice for controls. Measured substrate glycogen in the heart (FIG. 20A), quad (FIG. 20B), diaphragm (FIG. 20C), triceps (FIG. 20D), and psoas (FIG. 20E) illustrate a significant reduction in glycogen in aged GaaKO mice after 6 weekly doses at 40 mg/kg performed at 2 weeks post last dose and in-life tests of grip strength performed during the course of treatment (data not shown).

1. A composition comprising acid alpha glucosidase, wherein at least 50% of the total N-linked oligosaccharides of the acid alpha glucosidase are phosphorylated.

2. The composition of claim 1, wherein at least 25% of the N-linked oligosaccharides on acid alpha-glucosidase are bis-phosphorylated.

3. The composition of claim 1, wherein at least 50% of the N-linked oligosaccharides on acid alpha-glucosidase are bis-phosphorylated.

4. The composition of claim 1, wherein on average each acid alpha glucosidase molecule comprises at least 2 bis-phosphorylated N-linked glycans.

5. The composition of claim 1, wherein less than 8% of the acid alpha glucosidase comprises non-phosphorylated N-linked glycans.

6. The composition of claim 1, wherein less than 40% of the acid alpha glucosidase glycans comprises complex type N-glycans.

7. The composition of claim 1, wherein less than 3% of the acid alpha glucosidase glycans comprises hybrid type N-glycans.

8. The composition of claim 1, wherein the composition comprises less than 5 nanomolar affinity for CIMPR.

9. The composition of claim 1, wherein the composition comprises from about 0.1 to 5-fold greater ability to reduce glycogen compared to alglucosidase alfa.

10. The composition of claim 1, wherein the composition comprises from about 0.1 to 5-fold greater ability to reduce glycogen compared to alglucosidase alfa in at least one tissue selected from the group consisting of heart, quad, triceps, diaphragm, or psoas.

11. The composition of claim 1, wherein the acid alpha glucosidase is capable of being endogenously hydrolyzed.

12. A method of treating Pompe disease, the method comprising administering to a subject an effective amount of the composition of claim 1.

13. The method of claim 12, wherein administering comprises enzyme replacement therapy.
